(19) United States
(12) Patent Application Publication
(10) Pub. No.: US 2004/0033262 A1
(43) Pub. Date: Feb. 19, 2004

(54) Sustained release pharmaceutical composition of a cephalosporin antibiotic

(75) Inventors: Rajesh Suresh Kshirsagar, Chennai (IN); Sanjay Parbhatrao Boldhane, Chennai (IN); Kour Chand Jindal, Chennai (IN)

Correspondence Address: OLIFF & BERRIDGE, PLC, P.O. BOX 19928, ALEXANDRIA, VA, 22320, US

(73) Assignee: Orchid Health Care, Chennai (IN)

(21) Appl. No.: 10/222,930

(22) Filed: Aug. 19, 2002

(51) Int. Cl. A61K 31/545; A61K 31/736; A61K 9/20; A61K 9/22

(52) U.S. Cl. 424/468; 514/54; 514/200

(57) ABSTRACT

This invention relates to a sustained release pharmaceutical composition comprising at least a cephalosporin antibiotic, a mixture of polymers and other pharmaceutically acceptable excipients; in the composition, polymers are selected from mixture of galactomannans and neutral swellable polymers, which releases the active ingredient in a predetermined manner.

### FIELD OF THE INVENTION

This invention relates to a sustained release pharmaceutical composition comprising at least a cephalosporin antibiotic, a mixture of polymers and other pharmaceutically acceptable excipients. The polymers are selected from mixture of galactomannans and neutral swellable polymers which releases the active ingredient in a predetermined manner, the said galactomannans being selected from the group consisting of xanthan gum and neutral swellable polymer selected from the group consisting of poly (ethyl acrylate: methyl methacrylate) 2:1.

### BACKGROUND OF THE INVENTION

While many compounds are known to be useful as pharmacologically active substances, some of them have relatively short biological half life and needs to be administered several times a day in order to achieve desired therapeutic effects. Especially, the drugs used in treatment of microbial infections are required to be given more than once during a dosage regimen.

In an anti-microbial therapy, the main requirement is to maximize the blood concentration, preferably several folds higher than the minimum inhibitory concentration (MIC) for the active agent, yet to minimize both the risks of toxicity to the patient and of promoting microbial resistance. Although, oral administration will be the preferred route, in the case of antibiotics this route is frequently unattractive because of their low or variable oral bioavailability. In addition, extremely high plasma concentrations of antibiotics are frequently required to achieve the MIC values towards certain gram-negative bacteria (antibiotics and chemotherapy; anti-infective agents and their use in therapy 7^{th} edition Ed. By O'grady F, Finch R G, Lambert H P; Greenwood D; Churchill Livingstone 1997).

Sustained release preparation of drugs are advantageous as the administration frequency can be reduced by maintaining a constant plasma concentration of drug over an extended period of time to ensure sustained effect of active ingredient well above the MIC levels. In addition, these preparations are also expected to decrease side effects by suppressing the rapid rise in the blood levels of the drug.

This has been primarily achieved by development of a novel drug delivery system utilizing diverse techniques and principles. Amongst these, known in the art is one such delivery system, which employs hydrophilic polymers to sustained or modified release pharmaceutical composition. In the modified release solid dosage forms comprising a drug, such drug is dispersed uniformly in a mixture of polymers and the release of the drug is controlled primarily by diffusion of the drug, or by surface erosion of the hydrophilic polymers into the surrounding medium or by a combination of both processes. Control of the rate of release can produce constant blood levels of the active ingredient that may result in reducing the frequency of administration, thereby improving patient compliance to the dosage regimen.

The relevant prior art methods, which teach adaptation of diverse delivery system for the sustained release of the active ingredient, are as follows:

U.S. Pat. No. 6,120,803 discloses an active agent dosage form, which is adapted for retention in the stomach and useful for the prolonged delivery of an active agent to a fluid environment. The active agent dosage form is a polymer composition that swells upon contact with the fluid of the stomach. A portion of the polymer composition is surrounded by a band of insoluble material that prevents the covered portion of the polymer composition from swelling and provides a segment of the dosage form that is of sufficient rigidity to withstand the contraction of the stomach and delay expulsion of the dosage form from the stomach until substantially all the active agent has been dispensed.

U.S. Pat. No. 5,128,142 discloses a controlled release formulation comprising an absorbate of a mixture of pharmaceutically active ingredients and an inactive substance absorbed on a cross linked polymer. The inactive substance is selected to modify the dissolution of active ingredients from the cross linked polymer in vivo. The inactive substance is preferably present in the absorbate in an amount of 0.5-3 parts by weight relative to 1 part by weight of the active ingredients.

U.S. Pat. No. 4,968,508 discloses a sustained release matrix tablet comprising from about 0.1% to about 90% by weight of Cefaclor, about 5% of about 29% by weight by hydrophilic polymer and about 0.5% to about 25% by weight of an acrylic polymer which dissolve at a pH in the range of about 5.0 to about 7.4, the total weight of polymers being less than 30% by weight of the formulation. Although a specific Cefaclor formulation is claimed the text suggests that the matrix formulation is suitable for weakly basic drugs and particularly suitable for Cephalexin and Cefaclor.

International Publication number WO 02/41876 discloses a pharmaceutical composition in the form of a tablet for controlled release of an active ingredient comprising a cephalosporin antibiotic such as Cephalexin, Cefaclor or their pharmaceutically acceptable hydrates, salts or esters as active ingredients, and a mixture of hydrophilic polymers selected from the group consisting of at least one sodium alginate and at least one xanthan gum as controlled release matrix and optionally probenecid as an antibiotic adjuvant as either immediate release or controlled release part. The composition may contain one or more of a water-soluble and/or water dispersible diluent. The quantity of the hydrophilic polymers matrix still provides the desired once a day profile.

International Publication number WO 02/36126 discloses a fast disintegrating controlled release oral composition comprising a core material containing Cefuroxime Axetil present as controlled release form, the Cefuroxime axetil-being provided with an outer coating of a copolymer selected from aqueous dispersions of enteric methacrylic acid and methacrylic acid esters anionic copolymers having carboxyl group as the functional group or mixture thereof and an inner coating of a sustained release copolymer selected from aqueous dispersions of acrylate or methacrylate pH independent copolymers having quaternary ammonium group as a functional group or mixture thereof. Additionally the coating composition may contain plasticizers. The composition is suitable for once daily administration.

U.S. Pat. No. 4,250,166 discloses a long acting Cephalexin preparation comprising of normal quick releasing Cephalexin and particulate Cephalexin coated with a copolymer of methacrylates and methacrylic acid which dissolves at pH from 5.5 to 6.5 and the potency ratio of the normal Cephalexin to coated Cephalexin is between 40:60 and 25:75.

U.S. Pat. No. 6,399,086 discloses a controlled release cephalosporin antibiotic agent preferably amoxicillin trihydrate in a hydrophilic and/or hydrophobic polymer matrix such that at least 50% but not more than 67.61±5.78% of the active agent is released within 3 to 4 hrs from oral administration and remainder is released at a controlled rate from the said composition. The composition teaches the use of commonly used hydrophilic polymers such as hydrophilic cellulose derivatives, hydrophilic methacrylic acid derivatives, chitosan, and alginates. Preferably, use of hydrophilic cellulose derivative such as methylcellulose, hydroxypropyl methycellulose, hydroxyethyl cellulose is cited.

The hydrophobic polymers used in the invention are acrylamides and polyamido derivatives and hydrophobic methacrylic acid derivatives. The preferred hydrophobic polymer is ethyl cellulose.

However with this profile wherein 67.61±5.78% of the drug is already out of the matrix, and considering a very short half-life of most cephalosporins such as Cephalexin and Cefprozil (55 min and 70 min) respectively the drug profile achieved cannot be suitable for once daily administration. Hence, it is necessary that the matrix formulation should release the drug over extended period of time.

The corresponding International Publication number WO 98/22091 discloses a controlled release oral drug delivery system comprising as active ingredient a betalactam antibiotic having a specific absorption site in a small intestine in a hydrophilic and/or hydrophobic polymer matrix such that at least 50% of the active agent is released within 3 to 4 hrs from oral administration and remainder is released at a controlled rate from the said composition.

U.S. Pat. No. 6,083,532 discloses a sustained release tablet comprising a drug to be released at a controlled rate and a sustained release formulation comprising atleast three different type of polymers including a pH dependent gelling polymer, a pH independent gelling polymer and an enteric polymer. The pH dependent gelling polymer comprises at least one of an alginate, a carboxyvinyl polymer, or a salt of carboxymethyl cellulose. The pH independent gelling polymer comprises at least one of a HPMC, HPEC, a HPC, a HEC, a methylcellulose, a xanthan gum or a polyethylene oxide. The enteric polymer comprises at least one of a polyacrylate material, a cellulose acetate phthalate, cellulose phthalate hydroxy propyl methyl ether, a polyvinyl acetate phthalate, a hydroxy propyl methyl cellulose acetate succinate, a cellulose acetate trimelliatate or shellac.

U.S. Pat. No. 5,948,440 discloses a controlled release tablet of an active ingredient comprising of Cefaclor, Cephalexin or their pharmaceutically acceptable hydrates, salts or esters as active ingredients and a mixture of hydrophilic polymers selected from the group consisting of at least one hydroxy propyl methylcellulose and at least one hydroxyl propyl cellulose. The composition optionally also contains one or more of a water soluble or water dispersible diluent, the quantities are such that the therapeutically effective active ingredient is released at a rate for twice daily administration of the pharmaceutical composition.

Japanese Patent JP 57165392A discloses a long acting Cephalexin tablet comprising Cephalexin mixed with ≧10% w/w oils and fats (e.g. higher fatty acid, higher alcohol, alcohol ester etc) and with a vehicle such as magnesium stearate and the mixture is pressed, formed into granules passing through a 20 mesh sieve, and subjected to the slug formed process to obtain a high quality long acting tablets. The rate of dissolution of Cephalexin can be controlled by selecting the kind of oils and fats and the number of the time of slug formation process.

International Publication number WO 00/15198 teaches controlled delivery pharmaceutical composition having temporal and spatial control, comprising a drug, a gas generating component a swelling agent, a viscolyzing agent and optionally a gel forming polymer. The viscolyzing agent initially and the gel forming polymer thereafter form a hydrated gel matrix which entraps the gas, causing the tablet to float so that it is retained in the stomach thereby providing spatial control and at the same time resulting in sustained release of the drug providing temporal control.

The combination of gas generating component, swelling agent and viscolyzing agent results in the controlled drug delivery systems. Thus all these components are essential for achieving the temporal and spatial control. A preferred once daily Ciprofloxacin formulation comprising 69.9% Ciprofloxacin base, 0.34% sodium alginate, 1.03% xanthan gum, 13.7% sodium bicarbonate, 12.1% cross linked polyvinyl pyrrolidone and optionally other excipients is disclosed.

Sustained release preparation of drugs are advantageous in the administration, frequency can be reduced by maintaining a constant plasma concentration of drug over an extended period of time to ensure sustained effect of active ingredient.

Since the antibiotics are high frequency/high dosing, extended release drug delivery systems have not been very successful in reducing the frequency. The present invention is based on the observation that the release of active ingredient from the delivery system is controlled by the specific polymers present in the matrix and in specific concentrations, thus allowing blood levels above MIC over extended period of time such that the frequency of the dosage form can be reduced to twice daily or once daily.

### OBJECTIVES OF THE INVENTION

The main object of the present invention is to provide a sustained release of the active ingredient from the pharmaceutical composition, which has blood levels above MIC over extended period of time.

Another objective of the present invention is to provide a sustained release pharmaceutical composition suitable for twice daily or once daily dosage form.

Yet another objective of the present invention is to provide a sustained release pharmaceutical composition, which releases the active ingredient in a predetermined manner.

Yet another objective of the present invention is to provide sustained release pharmaceutical composition of a cephalosporin antibiotic.

### SUMMARY OF THE INVENTION

Accordingly, the present invention relates to a sustained release pharmaceutical composition comprising at least a cephalosporin antibiotic, a mixture of galactomannans and neutral swellable polymers and other pharmaceutically acceptable excipients.

The polymers are selected in such a way to give sustained release of the active ingredient in a predetermined manner.

Preferably, the invention relates to sustained release pharmaceutical composition comprising a cephalosporin antibiotic, a mixture of galactomannans and neutral swellable polymers, said galactomannans being selected from the group consisting of xanthan gum and neutral swellable polymer selected from the group consisting of poly (ethyl acrylate: methyl methacrylate) 2:1.

More preferably, the present invention relates to the sustained release pharmaceutical composition which comprises about 30% to about 90% by weight of a cephalosporin antibiotic; about 2% to about 30% by weight of said mixture of polymers being selected from group consisting of about 0.1% to about 15% by weight of galactomannans, about 0.1% to about 15% of neutral swellable polymer by weight of sustained release composition.

Still more preferably, the present invention relates to the sustained release pharmaceutical composition comprises about 30% to about 90% by weight of cephalosporin antibiotic, about 2% to about 20% by weight of mixture of said polymers being selected from group consisting of galactomannans in an amount from about 0.1% to about 12% by weight and neutral swellable polymer in an amount from about 0.1% to about 12% by weight of sustained release composition.

According to yet another embodiment of the present invention, the sustained release pharmaceutical composition may be prepared by wet granulation method, the said method comprising steps of:

i. mixing an active ingredient, pharmaceutically acceptable excipients and galactomannans in a mixer,

ii. granulating the mixture of step (i) with a neutral swellable polymer,

iii. drying the granules of step (ii) by either tray drying or fluid bed drier,

iv. milling the dried granules of step (iii) followed by addition of a dry binder and a lubricant,

v. compressing the lubricated milled granules of step (iv) into tablets using a tablet press and if desired coating the tablets.

A binder such as low viscosity hydroxypropyl methyl cellulose (HPMC) can be added optionally, during wet granulation.

### DETAILED DESCRIPTION OF THE INVENTION

In an embodiment of the present invention, cephalosporin antibiotic used as active ingredient include Cephalexin, Cefprozil, Cefditoren pivoxil, Cefadroxil, Cefpodoxime proxetil, Cefuroxime axetil, Cefaclor, Cefamandole, Cefoxitin, Cephalothin, Cephaprin, Ceftizoxime, Cefonicid and their pharmaceutically acceptable hydrates, salts or esters.

In another embodiment of the present invention, cephalosporin antibiotic may be present in an amount from about 30 to about 90% by weight of the sustained release composition. Further, the cephalosporin antibiotic may be present in the amount from 100 mg to 2000 mg per dosage.

In an embodiment of the present invention, the galactomannans used is selected from the group consisting of xanthan gum, guar gum, locuat bean gum and the like. In an embodiment of the present invention, the neutral swellable polymer used is Poly (ethyl acrylate: methyl methacrylate) 2:1.

The composition optionally comprises of one or more excipients such as water soluble or water dispersible diluents, binders, lubricant wherein the quantities of galactomannans and neutral swellable polymers, and water soluble/water dispersible diluents are such that the therapeutically effective active ingredients is released at a rate suitable for once or twice daily administration of the dosage form.

The composition may contain one or more pharmaceutically acceptable diluent in amount about 1% to about 30% by weight of the total weight of composition.

These diluents may be water soluble or water dispersible. Examples of water soluble diluents that may be used in the present invention include lactose, mannitol, glucose, sorbitol, maltose, dextrates, dextrins and the like. Water dispersible diluents that may be used such as microcrystalline cellulose, pregelatinized starch, and the like. In the preferred embodiment, the diluent is lactose in amount from about 5% to about 20% by weight of the sustained release composition.

The composition may also contain a tablet binder at a concentration in the range of about 0.2% to about 12% by weight either alone or in combination of the total weight of composition. The binder that may be used includes polyvinyl pyrrolidone, hydroxypropyl methylcellulose, hydroxypropyl cellulose, gelatin, pregelatinized starch, sugar and the like.

The composition may also contain a tablet lubricant at a concentration in the range of about 0.2% to about 5% by weight either alone or in combination of the total weight of composition. The lubricant that may be used include, talc, stearic acid, magnesium stearate, colloidal silicon dioxide, calcium stearate, zinc stearate, hydrogenated vegetable oil and the like.

Xanthan gum is a high molecular weight, anionic, natural heteropolysaccharide gum produced by aerobic fermentation with the organism xanthomonas campestris. It contains D-glucose, D-mannose, D-glucuronate in the molar ratio of 2.8:2.0:20 and is partially acetylated with about 4.7% acetyl. Xanthan gum also includes about 3.0% pyruvate, which is attached to a single unit D-glucopyromosyl side chain as a metal. It dissolves in hot or cold water and the viscosity of aqueous solutions of xanthan gum is only slightly affected by changes in the pH of solution between 1 and 11.

Xanthan gum has a branched or helical configuration, thus results in excellent water wicking properties. When xanthan gum comes into contact with an aqueous medium of gastrointestinal tract, it hydrates and swells to form a gel. It has good swelling action on contact with an aqueous medium and overcomes the problem encountered by other gums, which either do not hydrate rapidly enough or hydrate too rapidly. Xanthan gum alone when used as a matrix forming agent in sustained release tablets, releases the drug slightly faster in acidic media, due to more rapid initial surface erosion than at higher pH. After hydration of the gum the drug release is essentially pH independent.

During the course of our studies, we found that when xanthan gum alone was used as matrix forming agent, the initial release of the cephalosporins was rapid, but the release retarded at later stage due to hydration of xanthan gum to forming a gel. The following tables disclose a typical composition and percent cephalexin released over a period of time.

| Ingredients | Weight (mg/tablet) | % w/w |
|---|---|---|
| Cephalexin | 795.32 | 75.74 |
| Lactose | 139.18 | 13.26 |
| Xanthan gum | 105.0 | 10.0 |
| Magnesium stearate | 10.5 | 1.0 |

| Time (hour) | Percent Cephalexin Released |
|---|---|
| 1 | 40.0 |
| 2 | 45.3 |
| 3 | 50.2 |
| 4 | 56.4 |
| 5 | 60.2 |
| 6 | 62.1 |

Xanthan gum polymers that may be used in the present invention include Xantural™ (Kelco), Rhodigel™ available from (Rhodia, USA) and xanthan gum (Jungbunzlauer, Austria.)

Poly (ethyl acrylate: methyl methacrylate) 2:1 is latex available only in aqueous dispersion either 30% or 40% solids including neutral emulsifier. It has no functional group and is practically neutral. Therefore, the films formed are insoluble in water or in aqueous medium over the entire pH range. The polymer swells in aqueous media and gives permeable membrane. The permeability is independent of pH.

We also found that when poly (ethyl acrylate: methyl methacrylate) 2:1 was used alone the release was retarded, however the tablet integrity was lost after 2 hours.

| Ingredients | Weight (mg/tablet) | % w/w |
|---|---|---|
| Cephalexin | 795.32 | 75.74 |
| Lactose | 139.18 | 13.26 |
| Eudragit NE 30D | 105.0 | 10.0 |
| Magnesium stearate | 10.5 | 1.0 |

| Time (hour) | Percent Cephalexin Released |
|---|---|
| 1 | 18.9 |
| 2 | 61.76 |
| 3 | 98.6 (Integrity lost) |

Poly (ethyl acrylate: methyl methacrylate) 2:1 is available under the brand name of Eudragit NE 30D from Rohm Pharma Company, Germany.

According to the present invention, when a pH independent neutral swellable polymer was mixed with xanthan gum along with active ingredient, the release from the composition was controlled initially and uniformly released over a period of time such that desired blood levels were achieved suitable for twice or once daily dosage form.

The combination of xanthan gum and poly (ethyl acrylate: methyl methacrylate) 2:1 is a unique combination suitable for sustained release of active ingredients, which are to be administered for once daily administration. Both the polymers give a pH independent release profile.

The granulation of the blend of active ingredient, diluent, binder, lubricant and xanthan gum with poly (ethyl acrylate: methyl methacrylate) 2:1 aqueous dispersions, form thinner but more effective film layers, where drug particles and granulating excipients are partially impregnated and during compression they are embedded in a fine network of thin polymer layers. When the tablet comes in contact with aqueous media of the gastrointestinal tract the thin film of poly (ethyl acrylate: methyl methacrylate) 2:1 controls the penetration of digestive fluids in to the composition and thus avoids initial erosion of xanthan gum, which is high at acidic pH. The poly (ethyl acrylate: methyl methacrylate) 2:1 slowly hydrates without disrupting the hydrophilic composition formed by the heteropolysaccharide. The insoluble poly (ethyl acrylate: methyl methacrylate) 2:1 forms a sponge like structure, which behaves as inert matrices. Once the xanthan gum is completely hydrated, it forms a gel and then, the release of active ingredient is governed by diffusion of dissolved drug through the pores, channels and capillaries of the insoluble polymer composition.

In the present invention, it was observed when these two polymers were mixed together in appropriate concentration to form a tablet, the release was controlled in such a manner that the dosage form is suitable for once daily administration.

We have found surprisingly when we control the release of the active ingredients so as to achieve a 14-16 hours release profile, we were able to achieve the blood levels suitable for once or twice daily dosage form.

The present invention is illustrated by the following examples, are not intended to be limited to the scope of the invention.

### BRIEF DESCRIPTION OF ACCOMPANYING DRAWING

FIG. 1 represents graphical representation of Comparative plasma profile of Cephalexin OD v/s Cephalexin capsules

### EXAMPLES

### General Procedure for the Preparation of Sustained Release Tablet

Cephalosporin antibiotic, galactomannans, lactose were screened through 30 mesh sieve and granulated by aqueous dispersions of Eudragit NE 30D. Granules were dried in either tray drier or fluidized bed drier. The dried granules were milled, followed by addition of dry binder such as low viscosity hydroxypropyl methylcellulose and lubricant magnesium stearate.

### Dissolution Method

For all the examples, the tablets were tested for Cephalexin or Cefprozil in 900 ml of 0.1N HCl for 1 hour, after which the dissolution media was changed to pH 6.8 phosphate buffer 900 ml. The tablets were placed in 40-mesh basket (USP Type I) and rotated at 100 rpm.

In the following examples, the composition and its dissolution profiles are given in a tabular form.

### Example 1

### Composition

| Ingredients | Weight (mg/tablet) | % w/w |
|---|---|---|
| Cephalexin | 798.15 | 75.74 |
| Lactose | 188.15 | 18.26 |
| Xanthan gum | 21.0 | 2.0 |
| Eudragit NE 30D | 31.5 | 3.0 |
| Magnesium stearate | 10.5 | 1.0 |

### Dissolution Profile

| Time (hour) | Percent Cephalexin Released |
|---|---|
| 1 | 19.25 |
| 2 | 26.44 |
| 4 | 44.0 |
| 6 | 59.57 |
| 8 | 70.4 |
| 10 | 78.5 |
| 12 | 81.9 |

### Example 2

### Composition

| Ingredients | Weight (mg/tablet) | % w/w |
|---|---|---|
| Cephalexin | 795.32 | 75.25 |
| Lactose | 107.68 | 10.26 |
| Xanthan gum | 31.5 | 3.0 |
| Eudragit NE 30D | 52.5 | 5.0 |
| HPMC E5 | 52.5 | 5.0 |
| Magnesium stearate | 10.5 | 1.0 |

### Dissolution Profile

| Time (hour) | Percent Cephalexin Released |
|---|---|
| 1 | 25.21 |
| 2 | 30.18 |
| 4 | 38.17 |
| 6 | 50.84 |
| 8 | 63.70 |
| 10 | 73.18 |
| 12 | 78.60 |
| 14 | 84.17 |

### Example 3

### Composition

| Ingredients | Weight (mg/tablet) | % w/w |
|---|---|---|
| Cephalexin | 795.32 | 75.24 |
| Lactose | 97.18 | 9.26 |
| Xanthan gum | 42.0 | 4.0 |
| Eudragit NE 30D | 52.5 | 5.0 |
| HPMC E5 | 52.5 | 5.0 |
| Magnesium stearate | 10.5 | 1.0 |

### Dissolution Profile

| Time (hour) | Percent Cephalexin Released |
|---|---|
| 1 | 22.42 |
| 2 | 30.25 |
| 4 | 41.62 |
| 6 | 48.33 |
| 8 | 54.54 |
| 10 | 60.70 |
| 12 | 66.30 |
| 14 | 71.80 |

### Example 4

### Composition

| Ingredients | Weight (mg/tablet) | % w/w |
|---|---|---|
| Cephalexin | 795.32 | 75.24 |
| Lactose | 86.68 | 8.26 |
| Xanthan gum | 52.5 | 5.0 |
| Eudragit NE 30D | 52.5 | 5.0 |
| HPMC E5 | 52.5 | 5.0 |
| Magnesium stearate | 10.5 | 1.0 |

### Dissolution Profile

| Time (hour) | Percent Cephalexin Released |
|---|---|
| 1 | 35.57 |
| 2 | 41.96 |
| 4 | 54.46 |
| 6 | 65.00 |

### Example 5

### Composition

| Ingredients | Weight (mg/tablet) | % w/w |
|---|---|---|
| Cephalexin | 795.32 | 75.24 |
| Lactose | 107.68 | 10.26 |
| Xanthan gum | 52.5 | 5.0 |
| Eudragit NE 30D | 42.0 | 4.0 |
| HPMC E5 | 42.0 | 4.0 |
| Magnesium stearate | 10.5 | 1.0 |

### Dissolution Profile

| Time (hour) | Percent Cephalexin Released |
|---|---|
| 1 | 26.3 |
| 2 | 31.5 |
| 4 | 39.8 |
| 6 | 47.7 |
| 8 | 51.2 |
| 10 | 56.1 |
| 12 | 62.9 |
| 14 | 69.87 |

### Example 6

### Composition

| Ingredients | Weight (mg/tablet) | % w/w |
|---|---|---|
| Cephalexin | 795.32 | 75.74 |
| Lactose | 107.68 | 9.26 |
| Xanthan gum | 42.0 | 4.0 |
| Eudragit NE 30D | 42.0 | 4.0 |
| HPMC E5 | 52.5 | 5.0 |
| Magnesium stearate | 10.5 | 1.0 |

### Dissolution Profile

| Time (hour) | Percent Cephalexin Released |
|---|---|
| 1 | 26.4 |
| 2 | 31.4 |
| 4 | 38.7 |
| 6 | 49.5 |
| 8 | 61.7 |
| 10 | 67.8 |
| 12 | 80.0 |
| 14 | 85.2 |

### Example 7

### Composition

| Ingredients | Weight (mg/tablet) | % w/w |
|---|---|---|
| Cefprozil | 1084.52 | 77.54 |
| Lactose | 118.48 | 8.46 |
| Xanthan gum | 56.0 | 4.0 |
| Eudragit NE 30D | 56.0 | 4.0 |
| HPMC E5 | 70.0 | 5.0 |
| Magnesium stearate | 14.0 | 1.0 |

### Dissolution Profile

| Time (hour) | Percent Cefprozil Released |
|---|---|
| 1 | 17.4 |
| 2 | 21.2 |
| 4 | 22.0 |
| 6 | 24.2 |
| 8 | 33.1 |
| 10 | 40.2 |
| 12 | 45.6 |
| 14 | 53.0 |

### Example 8

### Composition

| Ingredients | Weight (mg/tablet) | % w/w |
|---|---|---|
| Cefprozil | 1084.52 | 77.54 |
| Lactose | 90.48 | 8.46 |
| Xanthan gum | 42.0 | 3.0 |
| Eudragit NE 30D | 28.0 | 2.0 |
| HPMC E5 | 140.0 | 10.0 |
| Magnesium stearate | 14.0 | 1.0 |

### Dissolution Profile

| Time (hour) | Percent Cefprozil Released |
|---|---|
| 1 | 20.3 |
| 2 | 24.4 |
| 4 | 32.1 |
| 6 | 40.8 |
| 8 | 57.4 |
| 10 | 70.5 |
| 12 | 78.5 |
| 14 | 84.8 |

### Bioavility Studies

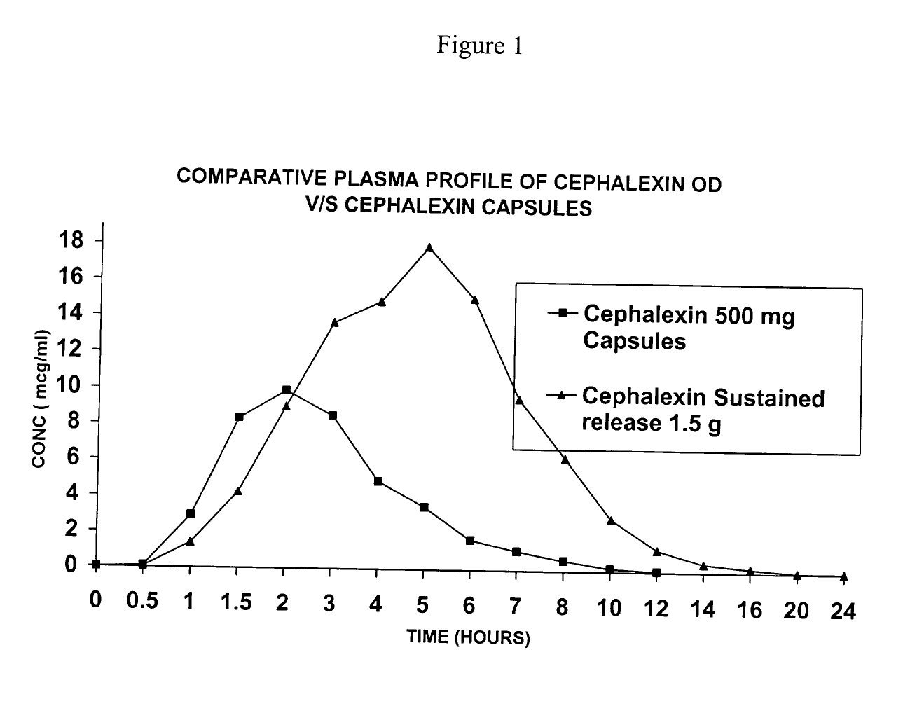

The bioavility study was conducted for comparison between conventional Cephalexin (500 mg) and sustained release composition formulation of Cephalexin 2 tablets of 750 mg, prepared according to the present invention. Eight healthy male volunteers were selected for the study in which each volunteer was administered a dose of the drug with 240 ml of water. The volunteers fasted overnight and had a standard breakfast before taking the drug. The desired blood levels upto 18 to 20 hours were achieved with the composition prepared according to the present invention, clearly indicating that in can used as once daily dosing. The data is summarized in table below. FIG. 1 shows a plot of Comparative plasma profile of Cephalexin OD v/s Cephalexin capsules.

| | | Cephalexin OD |
|---|---|---|
| Parameters | Cephalexin Capsules 500 mg | (750 mg × 2 T) |
| Cmax | 13.45 | 20.01 |
| (mcg/ml) | | |
| Tmax (hrs) | 2.18 | 4.87 |
| AUC 0-t mcg · h/ml | 34.62 | 100.34 |

The cephalosporin antibiotic exhibits minimal concentration dependent killing and produce short term or no persistent effect with most bacteria. The killing rate of these antibiotics saturates at concentrations of around 4 to 5 times the MBC, thus high concentration will not kill the bacteria faster than lower concentrations. It has also been suggested that a concentration much greater than the MIC decreases in bacterial kill potency. These findings have led to the hypothesis that continuously maintained concentrations above a certain level, related to the MIC would be more efficacious than the high peak through concentrations obtained with an intermittent dosing regimen. From the Pharmacokinetic data obtained, it is seen the sustained release formulation has achieved the Time/MIC equivalent to 3 times the dosing of conventional dosage regime which is essential for killing the bacteria.

While there have been shown and described what are believed to be the preferred embodiments of the present invention, it will be apparent to those of ordinary skill in the pharmaceutical formulating art that various modifications in the formulations and processes herein described can be made without departing from the scope of invention as it is defined by the appended claims.

1. A sustained release pharmaceutical composition comprising at least a cephalosporin antibiotic, a mixture of galactomannans and neutral swellable polymers and other pharmaceutically acceptable excipients.

2. The composition according to claim 1, which comprises about 30% to about 90% by weight of a cephalosporin antibiotic; about 2% to about 30% by weight of said mixture of polymers selected from about 0.1% to about 15% by weight galactomannans and about 0.1% to about 15% of neutral swellable polymer by weight of sustained release composition.

3. The composition according to claim 1, which comprises about 30% to about 90% by weight of cephalosporin antibiotic, about 2% to about 20% by weight of mixture of said polymers selected from galactomannans in an amount from about 0.1% to about 12% by weight and neutral swellable polymer in an amount from about 0.1% to about 12% by weight of sustained release composition.

4. The composition as claimed in claim 1, is in the form of a tablet.

5. The composition as claimed in claim 1, wherein the cephalosporin antibiotic is released at a rate suitable for once daily or twice daily administration.

6. The composition according to claim 1, wherein the cephalosporin antibiotic is selected from Cephalexin, Cefprozil, Cefditoren pivoxil, Cefadroxil, Cefpodoxime proxetil, Cefuroxime axetil, Cefaclor, Cefamandole, Cefoxitin, Cephalothin, Cephaprin, Ceftizoxime, Cefonicid and their pharmaceutically acceptable hydrates, salts or esters.

7. The composition according to claim 1, wherein the galactomannans used is selected from the group consisting of xanthan gum, guar gum or locuat bean gum.

8. The composition according to claim 1, wherein the excipients used are water soluble or water dispersible diluents, binders or lubricant.

9. The composition according to claim 8, wherein the water soluble or water dispersible diluent consisting of about 1 to 30% by weight of the composition.

10. The composition as claimed in claim 8, wherein the water soluble diluents used are lactose, mannitol, glucose, sorbitol, maltose, dextrates or dextrins.

11. The composition as claimed in claim 8, wherein the water dispersible diluents used is microcrystalline cellulose or pregelatinized starch.

12. The composition as claimed in claim 8, wherein the tablet binder concentration is in the range of about 0.2% to about 12% by weight either alone or in combination of the total weight of composition.

13. The composition as claimed in claim 8, wherein the binder is selected from polyvinyl pyrrolidone, hydroxypropyl methylcellulose, hydroxypropyl cellulose, gelatin, pregelatinized starch, sugar and like.

14. The composition as claimed in claim 8, wherein the lubricant concentration is in the range of about 0.2% to about 5% by weight either alone or in combination of the total weight of composition.

15. The composition as claimed in claim 8, wherein the lubricant used is selected from talc, stearic acid, magnesium stearate, colloidal silicon dioxide, calcium stearate, zinc stearate or hydrogenated vegetable oil.

16. A process for the preparation of the sustained release pharmaceutical composition, the said method comprising steps of:
i) mixing an active ingredient, pharmaceutically acceptable excipients and galactomannans in a mixer,
ii) granulating the mixture of step (i) with a neutral swellable polymer,
iii) drying the granules of step (ii) by either tray drying or fluid bed drier,
iv) milling the dried granules of step (iii) followed by addition of dry binder and a lubricant, and
v) compressing the lubricated milled granules of step (iv) into tablets using a tablet press and if desired coating the tablets with pharmaceutically acceptable agents.
